(19) United States
(12) Patent Application Publication
(10) Pub. No.: US 2024/0293583 A1
(43) Pub. Date: Sep. 5, 2024

(54) RADIOPAQUE CROSSLINKED ACRYLAMIDE COPOLYMERS AND USES THEREOF

(71) Applicant: Boston Scientific Scimed, Inc., Maple Grove, MN (US)

(72) Inventors: Heather Cirka, Hudson, MA (US); Yen-Hao Hsu, Shrewsbury, MA (US)

(73) Assignee: Boston Scientific Scimed, Inc., Maple Grove, MN (US)

(21) Appl. No.: 18/591,471

(22) Filed: Feb. 29, 2024

Related U.S. Application Data
(60) Provisional application No. 63/488,043, filed on Mar. 2, 2023.

(51) Int. Cl. A61K 49/04; A61K 45/06

(52) U.S. Cl. CPC A61K 49/0438; A61K 45/06; A61K 49/0419; A61K 49/0457

(57) ABSTRACT

The present disclosure provides radiopaque crosslinked acrylamide copolymers that comprise (a) a monofunctional acrylamide monomer containing one or more radiopaque atoms and/or a multifunctional acrylamide monomer containing one or more radiopaque atoms and (b) a monofunctional acrylamide monomer that does not contain one or more radiopaque atoms and/or a multifunctional acrylamide monomer that does not contain one or more radiopaque atoms, wherein the copolymer comprises at least one multifunctional acrylamide monomer. The present disclosure also provides radiopaque particulate compositions that comprise such radiopaque crosslinked acrylamide copolymers and medical procedures that comprise administering to a subject such radiopaque particulate compositions. The present disclosure further provides methods of making such radiopaque crosslinked acrylamide copolymers.

### CROSS-REFERENCE TO RELATED APPLICATIONS

This application claims the benefit of U.S. Provisional Patent Application Ser. No. 63/488,043 filed on Mar. 2, 2023, the disclosure of which is incorporated herein by reference.

### FIELD

The present disclosure relates to radiopaque crosslinked acrylamide copolymers, particulate compositions comprising radiopaque crosslinked acrylamide copolymers, and methods of making and using radiopaque crosslinked acrylamide copolymers and particulate compositions. The radiopaque crosslinked acrylamide copolymers and particulate compositions of the present disclosure are useful, for example, in various medical procedures.

### BACKGROUND

Polyacrylamide is a widely used hydrogel in the biomedical and biotechnology industries. Polyacrylamide hydrogel is currently used as the sole material component in the product the Bulkamid™. This product has been in medical use for a number of years as a bulking agent in cosmetic surgery and ophthalmic surgery and more recently has been used to treat stress urinary incontinence (SUI) in women. For this latter clinical case, the material remains permanently implanted to allow for relief of symptoms.

### SUMMARY

There is a need in the biomedical arts for radiopaque polyacrylamide materials, and for methods of making and using radiopaque polyacrylamide materials.

In some aspects, the present disclosure provides radiopaque crosslinked acrylamide copolymers that comprise (a) a monofunctional acrylamide monomer containing one or more radiopaque atoms and/or a multifunctional acrylamide monomer containing one or more radiopaque atoms and (b) a monofunctional acrylamide monomer that does not contain one or more radiopaque atoms and/or a multifunctional acrylamide monomer that does not contain one or more radiopaque atoms. The copolymer comprises at least one multifunctional acrylamide monomer selected from a multifunctional acrylamide monomer containing one or more radiopaque atoms and a multifunctional acrylamide monomer that does not contain one or more radiopaque atoms.

In some embodiments, the monofunctional acrylamide monomer that contains one or more radiopaque atoms comprises a single acrylamido moiety and one or more iodinated moieties.

In some embodiments, which can be used in conjunction with any of the above aspects and embodiments, the multifunctional acrylamide monomer that contains one or more radiopaque atoms comprises two three or four acrylamido moieties and one or more iodinated moieties.

In some embodiments, which can be used in conjunction with any of the above embodiments, the one or more iodinated moieties comprise an iodinated aromatic group.

In some embodiments, which can be used in conjunction with any of the above aspects and embodiments, the monofunctional acrylamide monomer containing one or more radiopaque atoms comprises a phenyl group substituted with (a) one, two, three, four or five iodine atoms, (b) one acrylamido moiety, and (c) optionally, one, two, three or four hydroxyl-containing groups selected from hydroxyl groups and/or C₁-C₄-hydroxyalkyl groups.

In some embodiments, which can be used in conjunction with any of the above aspects and embodiments, the multifunctional acrylamide monomer containing one or more radiopaque atoms comprises a phenyl group substituted with (a) one, two, three or four iodine atoms, (b) two, three, four or five acrylamido moieties, and (c) optionally, one, two or three hydroxyl-containing groups selected from hydroxyl groups and/or C₁-C₄-hydroxyalkyl groups.

In some embodiments, which can be used in conjunction with any of the above aspects and embodiments, the radiopaque crosslinked acrylamide copolymer is a hydrogel.

In some embodiments, which can be used in conjunction with any of the above aspects and embodiments, the radiopaque crosslinked acrylamide copolymer has a radiopacity that is greater than 100 Hounsfield units (HU).

In some aspects, the present disclosure provides radiopaque particulate compositions comprising radiopaque particles that comprise a radiopaque crosslinked acrylamide copolymer in accordance with any of the above aspects and embodiments.

In some embodiments, the radiopaque particulate composition further comprises one or more additional agents selected from the following: therapeutic agents, imaging agents, colorants, tonicity adjusting agents, suspension agents, wetting agents, and pH adjusting agents.

In some embodiments, which can be used in conjunction with any of the above aspects and embodiments, the radiopaque particulate composition is an injectable composition.

In some embodiments, which can be used in conjunction with any of the above aspects and embodiments, the radiopaque particulate composition is provided in a container in sterile dry form or in a suspension. For example, the container may be selected from a syringe, vial, ampoule or catheter component.

In some aspects, the present disclosure provides medical procedures that comprise administering to a subject a radiopaque particulate composition in accordance with any of the above aspects and embodiments.

In some embodiments, the medical procedure is selected from a tissue bulking procedure, a tissue spacing procedure, a blood vessel occlusion procedure, and a fiducial marking procedure.

In some embodiments, which can be used in conjunction with any of the above aspects and embodiments the particles are viewed under x-ray imaging during and/or after administration.

In some aspects, the present disclosure provides methods of making a radiopaque crosslinked acrylamide copolymer in accordance with any of the above aspects and embodiments, comprising photopolymerizing an aqueous solution that comprises (a) a monofunctional acrylamide monomer containing one or more radiopaque atoms and/or a multifunctional acrylamide monomer containing one or more radiopaque atoms, (b) a monofunctional acrylamide monomer that does not contain one or more radiopaque atoms and/or a multifunctional acrylamide monomer that does not contain one or more radiopaque atoms and (c) an one or more polymerization initiators, wherein the aqueous solution comprises at least one multifunctional acrylamide monomer selected from the multifunctional acrylamide monomer containing one or more radiopaque atoms and the multifunctional acrylamide monomer that does not contain one or more radiopaque atoms.

One advantage of the present disclosure is that polyacrylamide materials are provided that are visible with both MRI and x-ray imaging. This feature allows for a physician to confirm placement location during and after implantation.

The above and other aspects, embodiments, features and benefits of the present disclosure will be readily apparent from the following detailed description.

### BRIEF DESCRIPTION OF THE DRAWINGS

FIG. 1 schematically illustrates methods of forming radiopaque crosslinked acrylamide polymers in accordance with two embodiments of the present disclosure.

### DETAILED DESCRIPTION

In some aspects, radiopaque crosslinked acrylamide polymers are provided herein that comprise a monofunctional acrylamide monomer containing one or more radiopaque atoms and/or a multifunctional acrylamide monomer containing one or more radiopaque atoms.

The one or more radiopaque atoms may be selected, for example, from Br, I, Bi, Ba, Gd, Ta, Zn and Au, among others, thereby making the polymer more absorptive of x-rays and thus more visible under x-ray imaging techniques such as x-ray fluoroscopy and computerized tomography (CT) scanning.

As used herein, “polymers” are molecules that contain multiple copies of one or more types of constitutional species, also referred to as monomers. The number of monomers within a given polymer may vary widely, ranging, for example, from 5 to 10 to 25 to 50 to 100 to 1000 to 10,000 or more constitutional units. As used herein, the term “monomers” may refer to the free monomers and those that are incorporated into polymers (also referred to herein as monomer “residues”), with the distinction being clear from the context in which the term is used. “Copolymers” are polymers that contain two, three, or more different monomers.

In some embodiments, radiopaque crosslinked acrylamide polymers are provided that comprise (a) a monofunctional acrylamide monomer containing one or more radiopaque atoms and/or a multifunctional acrylamide monomer containing one or more radiopaque atoms and (b) a monofunctional acrylamide monomer that does not contain one or more radiopaque atoms and/or a multifunctional acrylamide monomer that does not contain one or more radiopaque atoms, wherein at least one multifunctional acrylamide monomer selected from the multifunctional acrylamide monomer containing one or more radiopaque atoms and the multifunctional acrylamide monomer that does not contain one or more radiopaque atoms is included to provide crosslinking.

As one example, radiopaque crosslinked acrylamide copolymers are provided that comprise (a) a monofunctional acrylamide monomer containing one or more radiopaque atoms and (b) a multifunctional acrylamide monomer containing one or more radiopaque atoms. As another example, acrylamide copolymers may comprise (a) a monofunctional acrylamide monomer containing one or more radiopaque atoms and (b) a multifunctional acrylamide monomer that does not contain one or more radiopaque atoms. As another example, the acrylamide copolymers may comprise (a) a monofunctional acrylamide monomer containing one or more radiopaque atoms, (b) a monofunctional acrylamide monomer that does not contain one or more radiopaque atoms and (c) a multifunctional acrylamide monomer containing one or more radiopaque atoms. As another example, the acrylamide copolymers may comprise (a) a monofunctional acrylamide monomer containing one or more radiopaque atoms, (b) a monofunctional acrylamide monomer that does not contain one or more radiopaque atoms and (c) a multifunctional acrylamide monomer that does not contain one or more radiopaque atoms. As another example, the acrylamide copolymers may comprise (a) a monofunctional acrylamide monomer containing one or more radiopaque atoms, (b) a multifunctional acrylamide monomer containing one or more radiopaque atoms and (c) a multifunctional acrylamide monomer that does not contain one or more radiopaque atoms. As another example, the acrylamide copolymers may comprise (a) a monofunctional acrylamide monomer that does not contain one or more radiopaque atoms, (b) a multifunctional acrylamide monomer containing one or more radiopaque atom and (c) a multifunctional acrylamide monomer that does not contain one or more radiopaque atoms. As a further example, the acrylamide copolymers may comprise (a) a monofunctional acrylamide monomer containing one or more radiopaque atoms, (b) a monofunctional acrylamide monomer that does not contain one or more radiopaque atoms, (c) a multifunctional acrylamide monomer containing one or more radiopaque atoms, and (d) a multifunctional acrylamide monomer that does not contain one or more radiopaque atoms.

The monofunctional acrylamide monomer that contains one or more radiopaque atoms comprises a single acrylamido moiety,

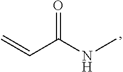

and one or more iodinated moieties. The multifunctional acrylamide monomer that contains one or more radiopaque atoms comprises two, three, four or more acrylamido moieties and one or more iodinated moieties.

In some embodiments, the iodinated moiety comprises an iodinated aromatic group (also referred to as an iodo-aromatic group). Examples of iodinated aromatic groups include iodine-substituted monocyclic aromatic groups and iodine-substituted multicyclic aromatic groups, such as iodo-phenyl groups and iodo-naphthyl groups. The iodinated aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.).

Examples of iodinated aromatic moieties include those that comprise one or more monocyclic or multicyclic aromatic structures, such as a benzene or naphthalene structure, substituted with (a) one or more iodine groups (e.g., one two, three, four, five, six or more iodine atoms) and (b) one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from amide groups, amine groups, ether groups, ester groups, or carbonate groups, among others.

Examples of iodinated aromatic moieties include those that comprise hydroxy-iodo-phenyl groups, C₁-C₄-hydroxyalkyl-iodo-phenyl groups, hydroxy-iodo-naphthyl groups, or C₁-C₄-hydroxyalkyl-iodo-naphthyl groups. Particular examples of hydroxyalkyl-iodo-phenyl groups include those selected from a mono-hydroxy-mono-iodo-phenyl group, a mono-hydroxy-di-iodo-phenyl group, a mono-hydroxy-tri-iodo-phenyl group, a mono-hydroxy-tetra-iodo-phenyl group, a di-hydroxy-mono-iodo-phenyl group, a di-hydroxy-di-iodo-phenyl group, a di-hydroxy-tri-iodo-phenyl group, a tri-hydroxy-mono-iodo-phenyl group, and a tri-hydroxy-di-iodo-phenyl group. Particular examples of C₁-C₄-hydroxyalkyl-iodo-phenyl groups include those selected from a mono-C₁-C₄-hydroxyalkyl-mono-iodo-phenyl group, a mono-C₁-C₄-hydroxyalkyl-di-iodo-phenyl group, a mono-C₁-C₄-hydroxyalkyl-tri-iodo-phenyl group, a mono-C₁-C₄-hydroxyalkyl-tetra-iodo-phenyl group, a di-C₁-C₄-hydroxyalkyl-mono-iodo-phenyl group, a di-C₁-C₄-hydroxyalkyl-di-iodo-phenyl group, a di-C₁-C₄-hydroxyalkyl-tri-iodo-phenyl group, a tri-C₁-C₄-hydroxyalkyl-mono-iodo-phenyl group, and a tri-C₁-C₄-hydroxyalkyl-di-iodo-phenyl group.

In various embodiments, the ring structures of the above phenyl and naphthyl groups may be directly substituted with one, two, three, four or more acrylamido moieties.

Examples of monofunctional acrylamide monomer containing one or more radiopaque atoms include N-(3-iodophenyl)-2-propenamide, N-(4-iodophenyl)-2-propenamide, N-(2-iodophenyl)-2-propenamide, and N-(2,4,6-triiodophenyl)-2-propenamide, among others.

Examples of multifunctional acrylamide monomers containing one or more radiopaque atoms include 1,3-bisacrylamido-2,4,6-triiodobenzene,

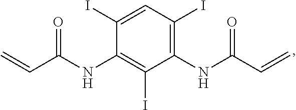 [SMILES: C=CC(=O)Nc1c(I)cc(I)c(NC(=O)C=C)c1I]

1,3-bisacrylamido-5-iodobenzene, and 1,3-bisacrylamido-2,4,5,6-tetraiodobenzene, among others.

Examples of monofunctional acrylamide monomers that do not contain one or more radiopaque atoms include acrylamide, N—C₁-C₆-alkylacrylamides, such as N-methylacrylamide, N-ethylacrylamide, N-n-propylacrylamide, N-isopropylacrylamide, etc., and C₁-C₆-alkylacrylamides, such as methacrylamide, ethacrylamide, n-propylacrylamide, isopropacrylamide, etc., among others.

Examples of multifunctional acrylamide monomers that do not contain one or more radiopaque atoms include, for example, N,N′-methylenebisacrylamide, N,N′-1,2-ethylenebisacrylamide, N,N′-1,3-trimethylenebisacrylamide, N, N′-1,4-tetramethylenebisacrylamide, N,N′-1,5-pentamethylenebisacrylamide, N,N′-1,7-pentamethylenebisacrylamide, N,N′-1,6-hexamethylenebisacrylamide, N,N′-1,8-octamethylenebisacrylamide, and N,N′-1,12-dodecamethylenebisacrylamide, among others, each of which can provide varying degrees of hydrophobicity.

In various embodiments, the radiopaque crosslinked acrylamide copolymer is a hydrogel. As used herein, a “hydrogel” is a crosslinked polymer that absorbs water but does not dissolve when placed in water.

Radiopaque crosslinked acrylamide copolymers in accordance with the present disclosure may be made, for example, by photopolymerization, e.g., using visible or ultraviolet light, of an aqueous solution containing a combination of comonomers as set forth above in the presence of one or more suitable initiators such as ammonium persulfate, riboflavin, and N,N,N⁰,N⁰-tetramethylene diamine (TEMED), and/or lithium phenyl-2,4,6-trimethylbenzoyl phosphate (LAP). Porosity of the resulting radiopaque crosslinked acrylamide copolymer may be varied, for example, by varying the ratio of mono-functional monomers to multi-functional comonomers and/or by varying the total concentration of comonomers in the solution.

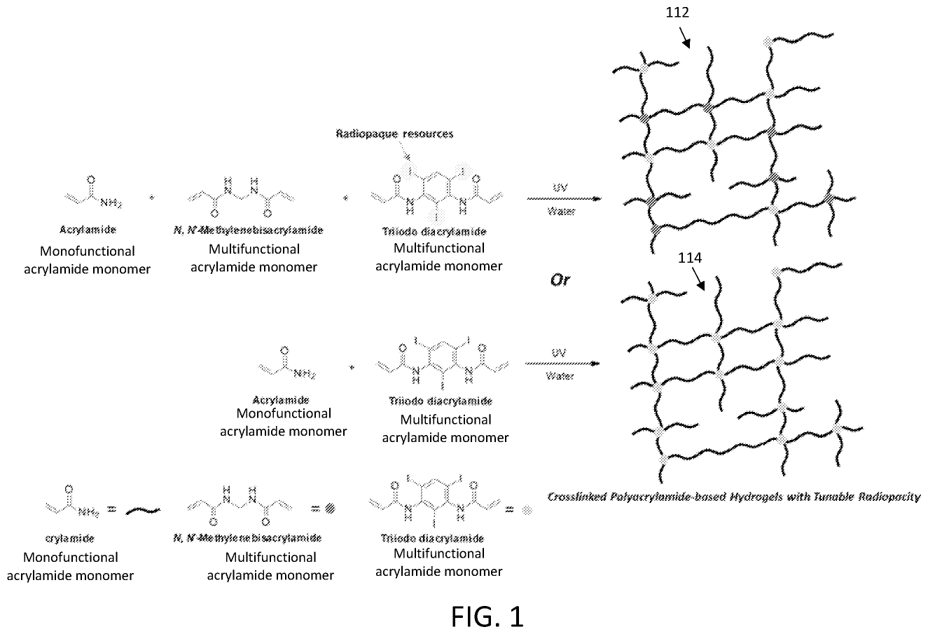

For example, reference to FIG. 1, in one embodiment, acrylamide, N,N′-methylenebisacrylamide, and a triiodo-diacrylamide monomer are crosslinked with ultraviolet light in an aqueous solution to form a radiopaque crosslinked acrylamide copolymer hydrogel 112. In another embodiment, acrylamide and a triiodo-diacrylamide monomer are crosslinked with ultraviolet light in an aqueous solution to form a radiopaque crosslinked acrylamide copolymer hydrogel 114.

The radiopaque crosslinked acrylamide copolymer of the present disclosure may be in any desired form, including a slab, a cylinder, a coating, and a particle (e.g., by emulsion polymerization or bulk polymerization/granulation). In some embodiments, the resulting radiopaque crosslinked acrylamide copolymer may be dried and then granulated into particles of suitable size. Granulating may be by any suitable process, for instance by grinding (including cryogrinding), crushing, milling, pounding, or the like. Sieving or other known techniques can be used to classify and fractionate the particles.

In various embodiments, the radiopaque crosslinked acrylamide copolymers in accordance with the present disclosure have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values).

Radiopaque crosslinked acrylamide copolymer particles formed using the above and other techniques may varying widely in size, for example, typically having an average size ranging from 1 to 1000 microns, more typically from 50 to 950 microns.

In various embodiments, compositions are provided which contain a radiopaque crosslinked acrylamide copolymer as described herein as well as one or more additional agents. Such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, anti-cancer drugs, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(III)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imagable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

In some embodiments, radiopaque particulate compositions are provided which contain radiopaque crosslinked acrylamide copolymer particles as described herein (also referred to as radiopaque particles) as well as one or more optional additional agents such as those described above (e.g., therapeutic agents, imaging agents, colorants, tonicity adjusting agents, suspension agents, wetting agents, pH adjusting agents, etc.). In particular embodiments, the radiopaque particulate compositions are injectable compositions.

Such radiopaque particulate compositions may be stored and transported in a sterile dry form. The dry composition may be shipped, for example, in a syringe, catheter, vial, ampoule, or other container, and it may be mixed with a suitable liquid carrier (e.g. sterile water for injection. physiological saline, phosphate buffer, a solution containing an imaging contrast agent, etc.) prior to administration. In this way the concentration of the radiopaque particles (and the one or more optional additional agents) in the administered composition may be varied at will, depending on the specific application at hand, as desired by the health care practitioner in charge of the procedure. One or more containers of liquid carrier may also be supplied and shipped, along with the dry radiopaque particles, in the form of a kit.

Such radiopaque particulate compositions may also be stored and transported in form of a sterile suspension that contains water in addition to the radiopaque particles and the one or more optional additional agents. The suspension may be stored, for example, in a syringe, catheter, vial, ampoule, or other container. The suspension may also be mixed with a suitable liquid carrier (e.g. sterile water for injection, physiological saline, phosphate buffer, a solution containing contrast agent, etc.) prior to administration, allowing the concentration of administered radiopaque particles (as well as other optional additional agents) in the suspension to be reduced prior to injection, if so desired by the health care practitioner in charge of the procedure. One or more containers of liquid carrier may also be supplied to form a kit.

In various embodiments, kits are provided that include one or more delivery devices for delivering the radiopaque particulate compositions to a subject.

The radiopaque particulate compositions described herein are visible under x-ray imaging techniques such as x-ray fluoroscopy and computerized tomography (CT) scanning, among others. In various embodiments, the radiopaque particulate compositions have a radiopacity that is greater than 100 Hounsfield units (HU), beneficially ranging anywhere from 100 HU to 250 HU to 500 HU to 750 HU to 1000 HU or more.

The radiopaque particulate compositions described herein can be administered to patients for achieving a number of medical outcomes. After administration, the compositions of the present disclosure can be imaged using a suitable imaging technique as previously noted.

For example, the radiopaque particulate compositions can be injected to provide spacing between tissues, the radiopaque particulate compositions can be injected (e.g., in the form of blebs) to provide fiducial markers, the radiopaque particulate compositions can be injected for tissue augmentation or regeneration, the radiopaque particulate compositions can be injected as a filler or replacement for soft tissue, the radiopaque particulate compositions can be injected to provide mechanical support for compromised tissue, the radiopaque particulate compositions be injected as a scaffold, and/or the radiopaque particulate compositions can be injected as a carrier of therapeutic agents in the treatment of diseases and cancers and the repair and regeneration of tissue, among other uses.

The radiopaque particulate compositions of the present disclosure may be used in a variety of medical procedures, including the following, among others: a procedure to implant a fiducial marker comprising the radiopaque particles, a procedure to implant a tissue regeneration scaffold comprising the radiopaque particles, a procedure to implant a tissue support comprising the radiopaque particles, a procedure to implant a tissue bulking agent comprising the radiopaque particles, a procedure to implant a therapeutic-agent-containing depot comprising the radiopaque particles, a tissue augmentation procedure comprising implanting the radiopaque particles, a procedure to embolize tissue, including benign tumors, malignant tumors and other abnormal tissue, a procedure to introduce the radiopaque particles between a first tissue and a second tissue to space the first tissue from the second tissue.

The radiopaque particulate compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The radiopaque particulate compositions may be injected for the permanent or temporary occlusion of blood vessels, and thus may be useful for managing various diseases and conditions. For example, the radiopaque particulate compositions may be used for the controlled, selective obliteration of the blood supply to benign and malignant tumors including treating solid tumors such as uterine fibroids, renal carcinoma, bone cancer, brain cancer, and liver cancer, among various others. The idea behind this treatment is that preferential blood flow toward a tumor will carry the embolization agent to the tumor thereby blocking the flow of blood which supplies nutrients to the tumor, causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the particulate composition.

Radiopaque particulate compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, chronic venous insufficiency, varicocele, pelvic congestion syndrome, gastrointestinal bleeding, renal bleeding, urinary bleeding, varicose bleeding, uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Radiopaque particulate compositions in accordance with the present disclosure may also be used in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. A radiopaque crosslinked acrylamide copolymer comprising (a) a monofunctional acrylamide monomer containing one or more radiopaque atoms and/or a multifunctional acrylamide monomer containing one or more radiopaque atoms and (b) a monofunctional acrylamide monomer that does not contain one or more radiopaque atoms and/or a multifunctional acrylamide monomer that does not contain one or more radiopaque atoms, wherein the copolymer comprises at least one multifunctional acrylamide monomer selected from the multifunctional acrylamide monomer containing one or more radiopaque atoms and the multifunctional acrylamide monomer that does not contain one or more radiopaque atoms.

2. The radiopaque crosslinked acrylamide copolymer of claim 1, wherein the monofunctional acrylamide monomer that contains one or more radiopaque atoms comprises a single acrylamido moiety, 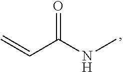
and an iodinated moiety.

3. The radiopaque crosslinked acrylamide copolymer of claim 2, wherein the iodinated moiety comprises an iodinated aromatic group.

4. The radiopaque crosslinked acrylamide copolymer of claim 1, wherein the multifunctional acrylamide monomer that contains one or more radiopaque atoms comprises two three or four acrylamido moieties and an iodinated moiety.

5. The radiopaque crosslinked acrylamide copolymer of claim 4, wherein the iodinated moiety comprises an iodinated aromatic group.

6. The radiopaque crosslinked acrylamide copolymer of claim 1, wherein the monofunctional acrylamide monomer containing one or more radiopaque atoms comprises a phenyl group substituted with (a) one, two, three, four or five iodine atoms, (b) one acrylamido moiety, and (c) optionally, one, two, three or four hydroxyl-containing groups selected from hydroxyl groups and/or C₁-C₄-hydroxyalkyl groups.

7. The radiopaque crosslinked acrylamide copolymer of claim 1, wherein the multifunctional acrylamide monomer containing one or more radiopaque atoms comprises a phenyl group substituted with (a) one, two, three or four iodine atoms, (b) two, three, four or five acrylamido moieties, and (c) optionally, one, two or three hydroxyl-containing groups selected from hydroxyl groups and/or C₁-C₄-hydroxyalkyl groups.

8. The radiopaque crosslinked acrylamide copolymer of claim 1, wherein the radiopaque crosslinked acrylamide copolymer is a hydrogel.

9. The radiopaque crosslinked acrylamide copolymer of claim 1, wherein the radiopaque crosslinked acrylamide copolymer has a radiopacity that is greater than 100 Hounsfield units (HU).

10. A radiopaque particulate composition comprising radiopaque particles that comprise a radiopaque crosslinked acrylamide copolymer that comprises (a) a monofunctional acrylamide monomer containing one or more radiopaque atoms and/or a multifunctional acrylamide monomer containing one or more radiopaque atoms and (b) a monofunctional acrylamide monomer that does not contain one or more radiopaque atoms and/or a multifunctional acrylamide monomer that does not contain one or more radiopaque atoms, wherein the copolymer comprises at least one multifunctional acrylamide monomer selected from the multifunctional acrylamide monomer containing one or more radiopaque atoms and the multifunctional acrylamide monomer that does not contain one or more radiopaque atoms.

11. The radiopaque particulate composition of claim 10, further comprising one or more additional agents selected from one or more of therapeutic agents, imaging agents, colorants, tonicity adjusting agents, suspension agents, wetting agents, and pH adjusting agents.

12. The radiopaque particulate composition of claim 10, wherein the radiopaque particles have an average size ranging from 50 to 950 microns.

13. The radiopaque particulate composition of claim 10, wherein the radiopaque particulate composition is an injectable composition.

14. The radiopaque particulate composition of claim 10, wherein the radiopaque particulate composition is provided in a container in sterile dry form.

15. The radiopaque particulate composition of claim 10, wherein the radiopaque particulate composition is provided in a container in a suspension.

16. The radiopaque particulate composition of claim 15, wherein the container is selected from a syringe, vial, ampoule or catheter component.

17. A medical procedure comprising administering to a subject a radiopaque particulate composition in accordance with claim 10.

18. The medical procedure of claim 17, wherein the medical procedure is selected from a tissue bulking procedure, a tissue spacing procedure, a blood vessel occlusion procedure, and a fiducial marking procedure.

19. The medical procedure of claim 17, wherein the particles are viewed under x-ray imaging during and/or after administration.

20. A method of making a radiopaque crosslinked acrylamide copolymer comprising photopolymerizing an aqueous solution that comprises (a) a monofunctional acrylamide monomer containing one or more radiopaque atoms and/or a multifunctional acrylamide monomer containing one or more radiopaque atoms, (b) a monofunctional acrylamide monomer that does not contain one or more radiopaque atoms and/or a multifunctional acrylamide monomer that does not contain one or more radiopaque atoms and (c) an one or more polymerization initiators, wherein the aqueous solution comprises at least one multifunctional acrylamide monomer selected from the multifunctional acrylamide monomer containing one or more radiopaque atoms and the multifunctional acrylamide monomer that does not contain one or more radiopaque atoms.
